(19) United States
(12) Patent Application Publication
(10) Pub. No.: US 2014/0012095 A1
(43) Pub. Date: Jan. 9, 2014

(54) STORAGE CONTROL APPARATUS, STORAGE CONTROL SYSTEM, AND STORAGE MEDIUM

(71) Applicant: SONY CORPORATION, Tokyo (JP)

(72) Inventors: YOICHIRO SAKO, Tokyo (JP); KOHEI ASADA, Kanagawa (JP); TAKATOSHI NAKAMURA, Kanagawa (JP); AKIRA TANGE, Tokyo (JP); KAZUYUKI SAKODA, Chiba (JP); KATSUHISA ARATANI, Kanagawa (JP); MITSURU TAKEHARA, Tokyo (JP); KAZUHIRO WATANABE, Tokyo (JP); HIROYUKI HANAYA, Kanagawa (JP); YUKI KOGA, Tokyo (JP); TOMOYA ONUMA, Shizuoka (JP)

(21) Appl. No.: 13/928,597

(22) Filed: Jun. 27, 2013

(30) Foreign Application Priority Data
Jul. 6, 2012 (JP) 2012-152087

(51) Int. Cl. A61B 7/02; A61B 1/00; A61B 5/00; A61B 1/04

(52) U.S. Cl. CPC A61B 7/023; A61B 1/041; A61B 1/00016; A61B 1/0002; A61B 5/7405
USPC 600/301; 600/586

(57) ABSTRACT

There is provided a storage control apparatus including a detection section which detects a body sound inside a body cavity, and outputs the body sound as an audio signal, and a storage control section which performs control in a manner that the audio signal output from the detection section is stored.

### BACKGROUND

The present disclosure relates to a storage control apparatus, a storage control system, and a storage medium.

While in the past stethoscopes have been used for listening to sounds within the body from outside of the body in order to examine such things as arrhythmia, heart murmurs and asthma, in recent years, endoscopes with a microphone attached have been proposed, which include mechanisms for detecting sounds at the distal end insertion portion or the like of the endoscope, and which detect sounds within the body along with an image of inside the body cavity.

Specifically, for example, JP 59-168832A discloses an endoscope apparatus which includes an optical fiber microphone mounted on an optical fiber, which guides light to the distal end insertion portion of an endoscope capable of imaging inside the body cavity. Further, JP H08-126603A discloses an endoscope apparatus which sends sound signals generated by a microphone, which is included at the distal end insertion portion of an endoscope, to a TV monitor, and outputs the sounds from the speakers of the TV monitor.

Further, JP 2001-104249A discloses an endoscope apparatus which is connected with a bone conduction type microphone capable of collecting sounds. Further, JP 2005-87297A discloses an endoscope apparatus which can accurately pick up vibrations of sound waves or the like inside the body cavity, by including a microphone at the distal end insertion portion.

Further, JP 2006-158515A discloses an endoscope apparatus which includes a microphone unit capable of attaching to and detaching from the distal end insertion portion of an endoscope.

### SUMMARY

Here, in all of the above described endoscope apparatuses, an imaging section which takes images of inside the body cavity is the main function, and a microphone which detects body sounds is subordinately included. Therefore, sound signals generated by the microphone are sent to an external apparatus (a television or the like which displays the captured image of inside the body cavity) as they are, and are simply output as sounds from the speakers of the external apparatus.

However, since body sounds are important factors in the judgment of diseases, an apparatus has been sought after which records (stores) more accurate body sounds, and effectively processes the body sounds so that they can be used for diagnosis, in order to accurately understand the condition of a disease and for early detection or the like of an abnormal part (affected part).

Accordingly, the present disclosure proposes a storage control apparatus, a storage control system, and a storage medium capable of more effectively acquiring body sounds used for diagnosis.

According to an embodiment of the present disclosure, there is provided a storage control apparatus including a detection section which detects a body sound inside a body cavity, and outputs the body sound as an audio signal, and a storage control section which performs control in a manner that the audio signal output from the detection section is stored.

According to an embodiment of the present disclosure, there is provided a storage control system including a transmission apparatus including a detection section which detects a body sound inside a body cavity, and outputs the body sound as an audio signal, and a transmission section which transmits the audio signal output from the detection section to an external apparatus after being temporarily stored, and a reception apparatus including a reception section which receives the audio signal from the transmission apparatus, and a storage control section which performs control in a manner that the audio signal received by the reception section is stored.

According to an embodiment of the present disclosure, there is provided a storage medium having a program stored thereon, the program causing a computer to function as a detection section which detects a body sound inside a body cavity, and outputs the body sound as audio signals, and a storage control section which performs control in a manner that the audio signal output from the detection section is stored.

According to the embodiments of the present disclosure described above, it becomes possible to more effectively acquire body sounds used for diagnosis.

### BRIEF DESCRIPTION OF THE DRAWINGS

FIG. 1 is a figure for describing an outline of a sound collection system according to a first embodiment of the present disclosure;

FIG. 2 is a block diagram which shows an analog configuration of a capsule type medical apparatus according to the first embodiment;

FIG. 3 is a block diagram which shows a digital configuration of a capsule type medical apparatus according to the first embodiment;

FIG. 4 is a block diagram which shows a configuration of a control apparatus according to the first embodiment;

FIG. 5 is a flow chart which shows the operation processes of the sound collection system according to the first embodiment;

FIG. 6 is a block diagram for describing another configuration of a signal processing section according to the first embodiment;

FIG. 7 is a block diagram which shows the main constituent elements of a capsule type medical apparatus performing a change of parameters based on an external control;

FIG. 8 is a figure which shows an example of an operation screen for instructing sound collection of a prescribed part;

FIG. 9 is a block diagram which shows the main constituent elements of a capsule type medical apparatus performing a change of parameters based on an internal control;

FIG. 10 is a block diagram which shows the main constituent elements of another capsule type medical apparatus performing a change of parameters based on an internal control;

FIG. 11 is a block diagram which shows the main constituent elements of a capsule type medical apparatus having plural types of microphones;

FIG. 12 is a block diagram which shows the main constituent elements of another capsule type medical apparatus having plural types of microphones;

FIG. 13 is a figure for describing the overall configuration of a sound collection system according to a second embodiment of the present disclosure;

FIG. 14 is a figure for describing an array signal process of a control apparatus according to the second embodiment;

FIG. 15 is a figure for describing an outline of a capsule type medical apparatus according to a third embodiment of the present disclosure;

FIG. 16 is a block diagram which shows the main constituent elements of the capsule type medical apparatus according to the third embodiment;

FIG. 17 is an explanatory diagram for describing a sound collection system according to a fourth embodiment of the present disclosure; and

FIG. 18 is an explanatory diagram for describing a sound collection system according to a fifth embodiment of the present disclosure.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The description will be given in the following order:

1. Outline of a Sound Collection System According to the Embodiments of the Present Disclosure

2. Each of the Embodiments

2-1. The first embodiment

(2-1-1) Configuration of the capsule

(2-1-2) Configuration of the control apparatus

(2-1-3) Operation processes

(2-1-4) Supplementation

2-2. The second embodiment

2-3. The third embodiment

2-4. The fourth embodiment

2-5. The fifth embodiment

3. Conclusion

### 1. OUTLINE OF A SOUND COLLECTION SYSTEM ACCORDING TO THE EMBODIMENTS OF THE PRESENT DISCLOSURE

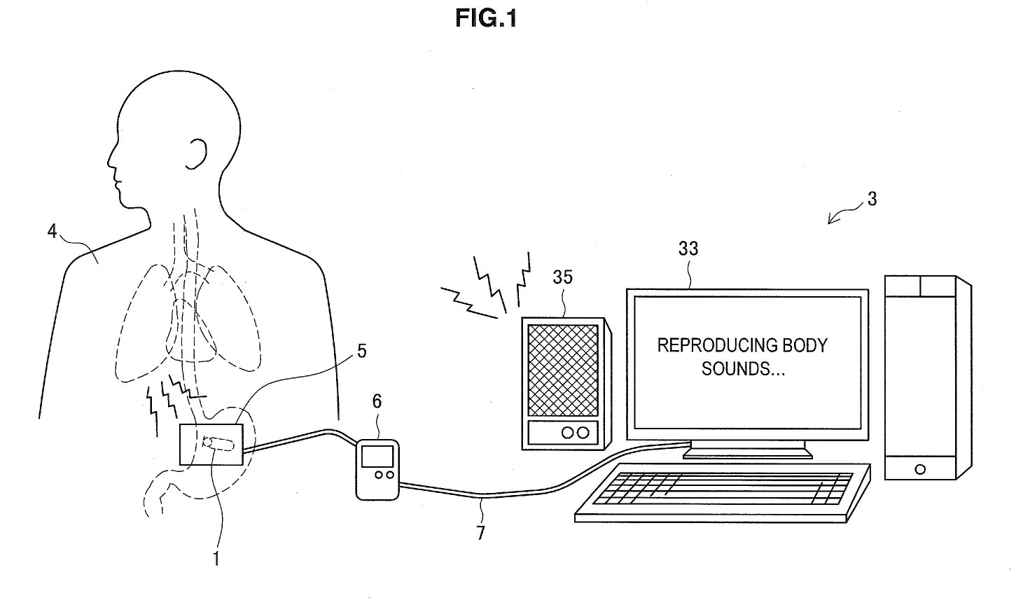

First, an outline of a sound collection system (storage control system) according to the embodiments of the present disclosure will be described with reference to FIG. 1. As shown in FIG. 1, the sound collection system according to the present embodiment has a capsule type medical apparatus 1 (hereinafter, called the capsule 1) introduced into the body by being swallowed or the like by a test subject 4, and a control apparatus 3. Further, the capsule 1 shown in FIG. 1 has a communication function, and is capable of performing transmission/reception of data with the external control apparatus 3.

For example, as shown in FIG. 1, in the case where an antenna 5 attached to an external surface of the body and an external unit 6 connected to the antenna 5 are placed on the test subject 4, the data transmitted from the capsule 1 introduced into the body cavity is received by the antenna 5, and is sent to the external unit 6. Note that in the example shown in FIG. 1, while the antenna 5 is attached to an external surface of the body in the vicinity of the stomach, the attachment point of the antenna 5 is not limited to the vicinity of the stomach, and may be attached, for example, to the external surface of the body corresponding to each part of the esophagus, bowels or the like by a plurality of antennas 5. Further, the antenna 5 capable of communication, even when the capsule 1 is positioned at any position inside the body cavity, may be attached to an external surface of the test subject 4 (or placed on a shield shirt worn by the test subject 4).

Also, the data sent from the antenna 5 to the external unit 6 is transmitted from the external unit 6 to the control apparatus 3. The external unit 6 and the control apparatus 3 may be connected to a freely attachable/detachable cable by a communication cable 7 such as a USB cable as shown in FIG. 1, or may be wirelessly connected. Operation buttons and a monitor are included on the front surface in the external unit 6. Further, the external unit 6 may be, for example, a user terminal such as a smart phone or a PDA (Personal Digital Assistant).

Further, the capsule 1 can receive data transmitted from the control apparatus 3 via the external unit 6 and the antenna 5.

Here, as described above, since body sounds are important factors in the judgment of diseases, an apparatus has been sought after which records (stores) more accurate body sounds, and effectively processes the body sounds so that they can be used for diagnosis, in order to accurately understand the condition of a disease and for early detection or the like of an abnormal part (affected part). In the present disclosure, body sounds are sounds generated within the body, and are, for example, a pulse, heart sounds, respiratory sounds, bowel sounds or the like.

Accordingly, the point of view of this situation led to creating the sound collection system according to the embodiments of the present disclosure. It is possible for the sound collection system according to the embodiments of the present disclosure to more effectively acquire body sounds used for diagnosis.

As shown in FIG. 1, body sounds collected inside the body cavity of a test subject 4 by the capsule 1 are temporarily stored as a transmission buffer, transmitted to the control apparatus 3 and output (reproduced) from a speaker 35 of the control apparatus 3, or are stored in a memory of the control apparatus 3. Further, it is possible for the control apparatus 3 to automatically diagnose an abnormal part of the heart, lungs, blood vessels or digestive tract, based on abnormal sounds such as arrhythmia, heart murmurs, wheezing or bowel sounds, and may display a diagnosis result on a display section 33.

In this way, according to the present embodiment, since body sounds inside the body cavity are collected, more accurate body sounds can be acquired than by a normal stethoscope which listens to body sounds from outside of the body by placing a chest piece on the body surface.

Further, a more suitable diagnosis can be performed by repeatedly listening to the body sounds, by storing the collected body sounds. Further, it becomes possible to continuously observe the body sounds surrounding an affected part, while the capsule 1 is stopped at a prescribed part. In addition, prescribed sounds from the body sounds collected by the capsule 1 may be transmitted to the control apparatus 3 upon being picked up. In this way, it becomes possible to pick up, for example, blood vessel sounds from the body sounds, and to observe the degree of turbulence within the blood vessels, or the degree of arteriosclerosis.

In this way, it becomes possible for early detection or the like of an abnormal part, by more effectively acquiring the body sounds used for diagnosis, and medical technology will be innovatively advanced.

Hereinafter, such a sound collection system according to the embodiment of the present disclosure will be described in detail by including a plurality of embodiments. Note that while a PC (Personal Computer) is shown in the example of FIG. 1 as an example of the control apparatus 3 according to the present embodiment, the control apparatus (reception apparatus) according to the embodiment of the present disclosure is not limited to this. For example, the control apparatus 3 according to the embodiment of the present disclosure may be a server, a smart phone, a PDA (Personal Digital Assistant), a notebook PC, a mobile phone, a portable music player, a mobile video processing apparatus, a portable game machine or the like. Further, in each embodiment described hereinafter, a capsule type medical apparatus will be used as an example of a storage control apparatus (transmission apparatus) according to the embodiment of the present disclosure.

### 2. EACH OF THE EMBODIMENTS

### 2-1. The First Embodiment

As shown in FIG. 1, the sound collection system according to a first embodiment of the present disclosure has a capsule 1 introduced into the body cavity of a test subject 4, and a control apparatus 3. Hereinafter, each of the constituent elements of the capsule 1 and the control apparatus 3 included in the first embodiment, and the operation processes of the sound collection system according to the first embodiment, will be sequentially described.

[2-1-1. Configuration of the Capsule]

The capsule 1, as described above, collects body sounds inside the body cavity of a test subject 4, temporarily stores the collected body sounds as a transmission buffer, and transmits the collected body sounds to an external apparatus.

Further, the capsule 1 may transmit the collected body sounds upon performing a prescribed signal process for the collected body sounds. For example, in the case where a prescribed part or internal organ is specified as an observation target of sounds, sounds originating from other parts or internal organs may become unnecessary noise. In order to improve this S/N ratio (signal/noise ratio), the capsule 1 may perform band restriction as a signal process for the collected body sounds.

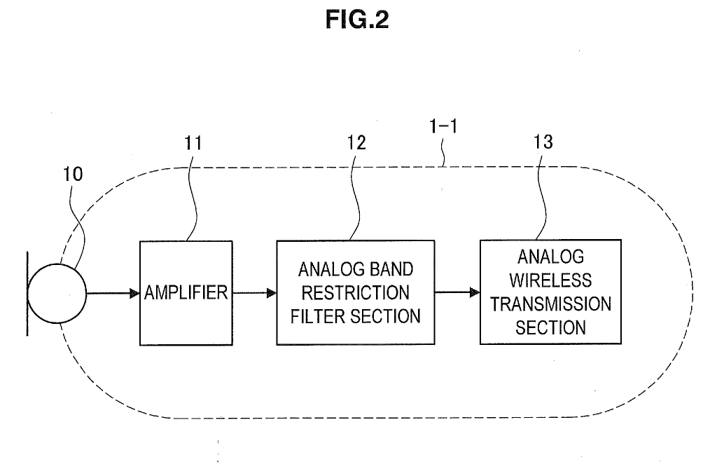

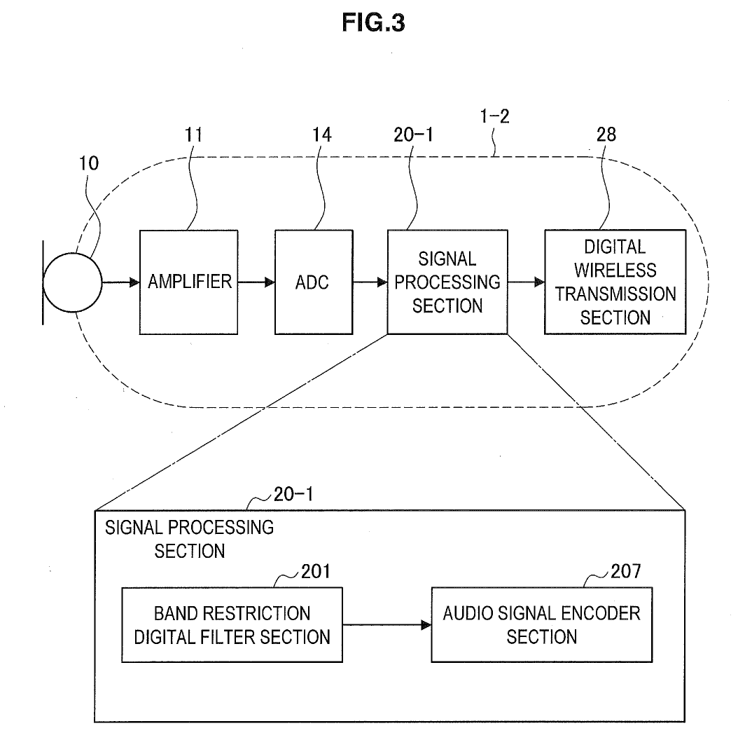

The band restriction according to the present embodiment may be implemented by an analog operation, or may be implemented by a digital operation. Hereinafter, a capsule 1-1 implemented by an analog operation, and a capsule 1-2 implemented by a digital operation, will be described with reference to FIGS. 2 and 3, respectively.

(Analog Configuration)

FIG. 2 is a block diagram which shows an analog configuration of the capsule 1-1 according to the first embodiment. As shown in FIG. 2, the capsule 1-1 has a microphone 10, an amplifier 11, an analog band restriction filter section 12, and an analog wireless transmission section 13.

The microphone 10 is a detection section which collects (detects) body sounds inside the body cavity, and outputs the body sounds as audio signals.

The amplifier 11 has a function which amplifies the audio signals output from the microphone 10.

The analog band restriction filter section 12 has a function which passes frequencies of prescribed bands, from among the audio signals output from the amplifier 11. In this way, the S/N ratio can be made large in the present embodiment. The analog band restriction filter section 12 may be implemented, for example, by a circuit such as a BPF (Band-Pass Filter), an LPF (Low-Pass Filter) or an HPF (High-Pass Filter).

The analog wireless transmission section 13 has a function which wirelessly transmits the audio signals which the analog band restriction filter section 12 has passed. The analog wireless transmission section 13 may be implemented, for example, by a circuit such as an AM (Amplitude Modulation), or an FM (Frequency Modulation).

Further, the analog wireless transmission section 13 according to the present embodiment has a transmission buffer (not shown in the figure) which temporarily stores transmission data (here, the audio signals).

(Digital Configuration)

Next, a configuration of the capsule 1-2 implemented by a digital operation will be described with reference to FIG. 3. As shown in FIG. 3, the capsule 1-2 has a microphone 10, an amplifier 11, an ADC (analog-digital convertor), a signal processing section 20-1, and a digital wireless transmission section 28. Since the microphone 10 and amplifier 11 have been described above with reference to FIG. 2, a description of them will be omitted here.

The ADC 14 is an electronic circuit which converts analog electrical signals into digital electrical signals. The ADC 14 converts the analog audio signals output from the amplifier 11 into digital audio signals, and outputs the digital audio signals.

The signal processing section 20-1 has a function which performs a prescribed signal process for the audio signals. The signal processing section 20-1 may be implemented, for example, by an operation apparatus such as a DSP (Digital Signal Processor), or an MPU (Micro-Processing Unit).

The signal processing section 20-1 according to the present embodiment, as shown in FIG. 3, functions as a band restriction digital filter section 201 and an audio signal encoder section 207. The band restriction digital filter section 201 has a function which passes frequencies of prescribed bands, from among the audio signals output from the ADC 14. Further, the band restriction digital filter section 201 performs digitalization with a BPF, LPF, HPF or the like, and it becomes possible for high precision control of a steep filter, direct phase filter or the like, which are difficult in analog, by performing digitalization with a band restriction filter.

The audio signal encoder section 207 (hereinafter, called the encoder section 207) has a function which encodes audio signals. The coding system is not particularly limited, and may be, for example, MP3 (MPEG Audio Layer-3), AAC (Advanced Audio Coding) or the like. Further, the coding system of the encoder section 207 may be a suitable coding system corresponding to a communication system of the digital wireless transmission section 28 of a later stage.

The digital wireless transmission section 28 has a function which wirelessly transmits the audio signals output from the signal processing section 20-1. The communication system of the digital wireless transmission section 28 is not particularly limited, and may be, for example, WiFi, Bluetooth, ZigBee or the like.

Further, the digital wireless transmission section 28 according to the present embodiment has a transmission buffer (not shown in the figure) which temporarily stores transmission data (here, the audio signals).

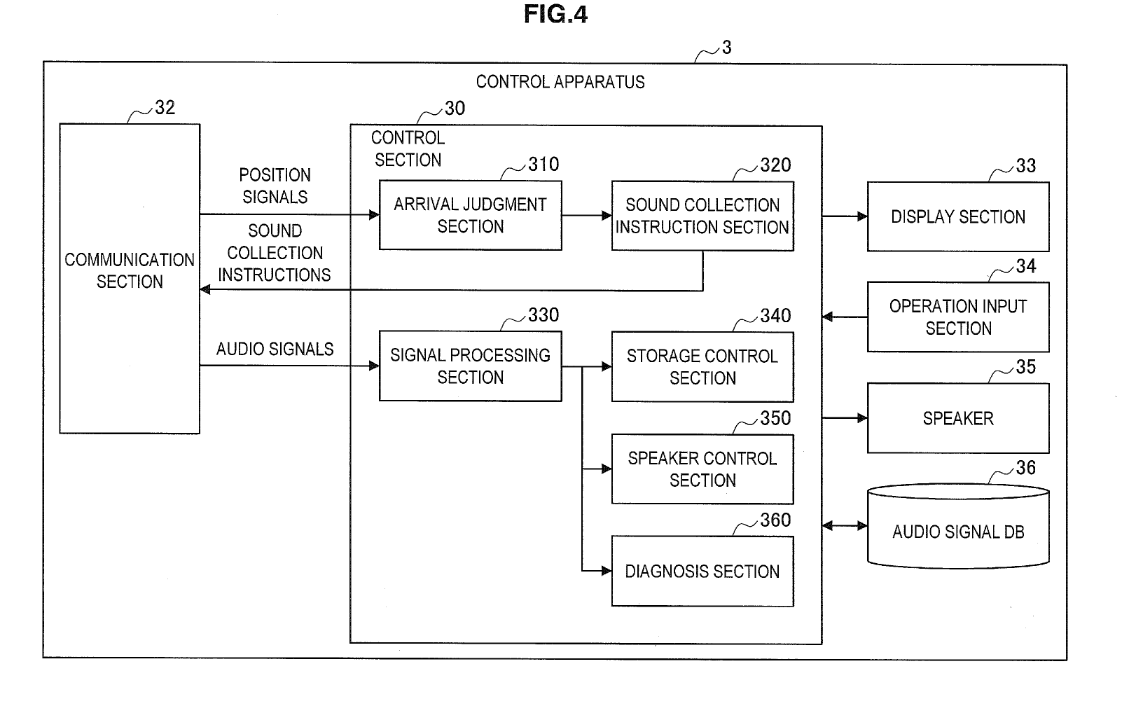

Heretofore, configurations of the capsule 1 according to the present embodiment have been specifically described. Note that the capsule 1 according to the present embodiment may continuously send signals (position signals) for present position detection of the capsule 1 inside the body cavity. To continue, a configuration of the control apparatus 3 according to the present embodiment will be described with reference to FIG. 4.

[2-1-2. Configuration of the Control Apparatus]

FIG. 4 is a block diagram which shows a configuration of the control apparatus 3 according to the first embodiment. As shown in FIG. 4, the control apparatus 3 according to the present embodiment has a control section 30, a communication section 32, a display section 33, an operation input section 34, a speaker 35, and an audio signal DB (database) 36.

(Communication Section)

The communication section 32 is connected to an external apparatus, and is an interface for performing transmission/reception of data. More specifically, the communication section 32 according to the present embodiment receives audio signals, present position information and the like from the capsule 1. Further, the communication section 32 may transmit control signals to the capsule 1.

(Control Section)

The control section 30 has a function which controls each constituent element of the control apparatus 3. More specifically, the control section 30 according to the present embodiment may function as an arrival judgment section 310, a sound collection instruction section 320, a signal processing section 330, a storage control section 340, a speaker control section 350, and a diagnosis section 360.

*Arrival Judgment Section

The arrival judgment section 310 detects the position of the capsule 1 based on a position signal transmitted from the capsule 1 when moving inside the body cavity, and judges whether or not the capsule 1 has reached the vicinity of a specific part set in advance. For example, the arrival judgment section 310 may detect the position of the capsule 1 based on the field intensity of a position signal received by the antenna 5. Note that the information for present position detection transmitted from the capsule 1 is not limited to a position signal, and may be, for example, a captured image of inside the body cavity imaged by an imaging section (not shown in the figure) of the capsule 1, or a sensor value or the like which is detected by various sensors (not shown in the figure) of the capsule 1.

*Sound Collection Instruction Section

The sound collection instruction section 320 carries out sound collection instructions to the capsule 1, in the case where it is judged by the arrival judgment section 310 that the capsule 1 has reached the vicinity of a specific part. Specifically, the sound collection instruction section 320 transmits control signals, which perform controls so as to collect sounds, from the communication section 32 to the capsule 1.

*Signal Processing Section

The signal processing section 330 performs a prescribed signal process for the audio signals transmitted from the capsule 1. Specifically, for example, in the case where the transmitted audio signals are encoded, a process is performed which decodes these audio signals, and extracts the original audio signals.

*Storage Control Section

The storage control section 340 performs control so as to store the audio signals output from the signal processing section 330 in the audio signal DB 36.

*Speaker Control Section

The speaker control section 350 performs control so as to output (reproduce) the audio signals output from the signal processing section 330 from the speaker 35.

*Diagnosis Section

The diagnosis section 360 has a function which analyses the audio signals output from the signal processing section 330 and performs a diagnosis. More specifically, for example, the diagnosis section 360 can detect abnormal sounds such as arrhythmia, heart murmurs, wheezing, or bowel sounds from the audio signals, and can judge an abnormal part of the heart, lungs, blood vessels or digestive tract. Further, a diagnosis result by the diagnosis section 360 may be displayed on the display section 33.

(Display Section)

The display section 33 has a function which performs screen display of operation screens, observation results of sounds, diagnosis results or the like, in accordance with the control of the control section 30. Note that the display section 33 may be implemented by an LCD (Liquid Crystal Display), an OLED (Organic Light-Emitting Diode), a CRT (Cathode Ray Tube) or the like.

(Operation Input Section)

The operation input section 34 has a function which detects operations by a user, and outputs input signals generated based on the detected operation input to the control section 30. The operation input section 34 may be implemented by a mouse, keyboard, touch panel or the like.

(Speaker)

The speaker 35 is an output apparatus which reproduces the body sounds, in accordance with the control of the speaker control section 350. Specifically, the speaker 35 reproduces the body sounds based on the audio signals.

(Audio Signal DB)

The audio signal DB 36 is a storage section which stores the audio signals, in accordance with the control of the storage control section 340.

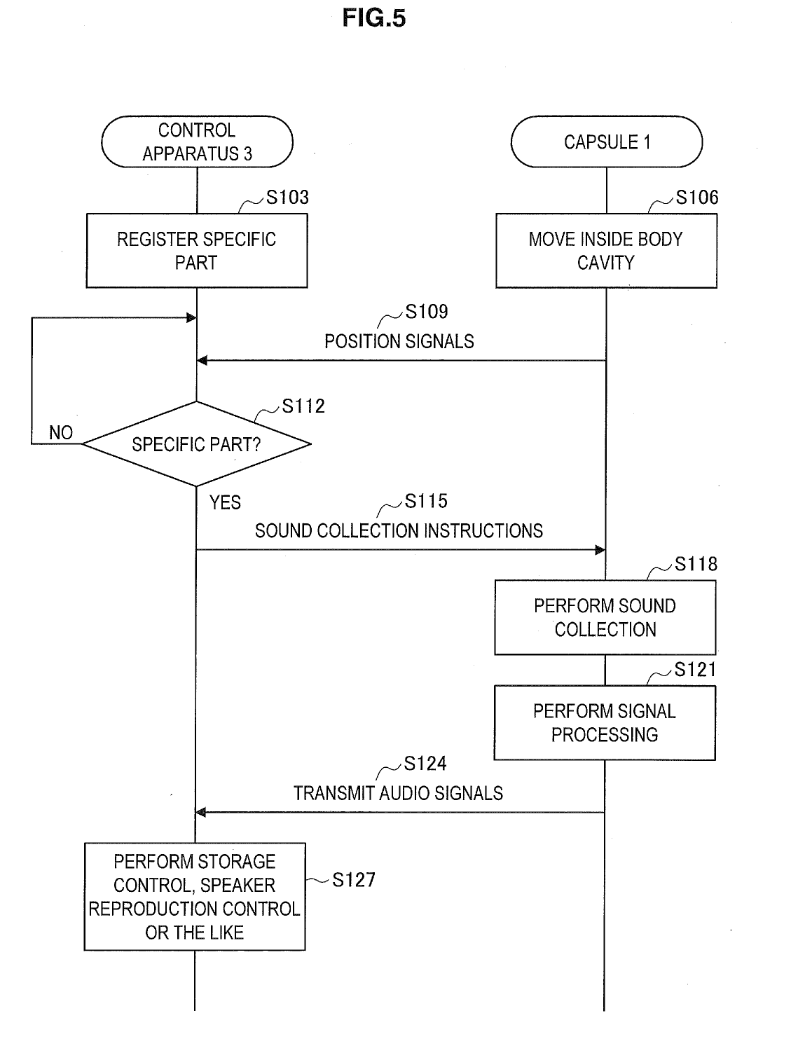

Heretofore, a configuration of the control apparatus 3 according to the first embodiment has been described in detail. To continue, the operation processes of the sound collection system according to the present embodiment will be described with reference to FIG. 5.

[2-1-3. Operation Processes]

FIG. 5 is a flow chart which shows the operation processes of the sound collection system according to the first embodiment. As shown in FIG. 5, first the control apparatus 3 registers a part targeted for sound collection (an observation target of sounds) as a specific part, in accordance with operations by a user such as a test subject 4 or medical staff (step S103).

Next, the capsule 1 introduced into the body cavity, by being swallowed or the like by the test subject 4, moves inside the body cavity by peristaltic movement (step S106).

Next, the capsule 1 continuously transmits a position signal for detecting the position of the capsule 1 to the control apparatus 3 while moving inside the body cavity (step S109).

Next, the arrival judgment section 310 of the control apparatus 3 detects the position of the capsule 1, based on the position signal transmitted from the capsule 1, and judges whether or not the capsule 1 has reached the vicinity of the specific part registered in advance (step S112).

Next, in the case where the capsule 1 has reached the vicinity of the specific part (step S112/Yes), the sound collection instruction section 320 performs control so as to collect sounds for the capsule 1 (step S115). Further, the capsule 1 may also perform control so as to temporary store the audio signals of the collected body sounds in a transmission buffer.

Next, the capsule 1 collects body sounds by the microphone 10 (step S118), and performs a prescribed signal process (step S121).

To continue, the capsule 1 transmits the audio signals, to which the signal process is performed, to the control apparatus 3 (step S124).

Then, the control apparatus 3 performs control so as to store the audio signals acquired from the capsule 1 in the audio signal DB 36, or performs control so as to reproduce the audio signals as body sounds from the speaker (step S127). Further, the control apparatus 3 may perform an automatic diagnosis based on the audio signals acquired from the capsule 1.

According to the present embodiment as described above, more accurate body sounds can be collected inside the body cavity. Further, according to the present embodiment, it becomes possible to perform a diagnosis based on more accurate body sounds.

[2-1-4. Supplementation]

Heretofore, the sound collection system according to the first embodiment has been described in detail. Note that each constituent element of the sound collection system according to the present embodiment is not limited to the above described constituent elements. Hereinafter, a supplementation of the present embodiment will be described.

(NR and D-Range Control Processes)

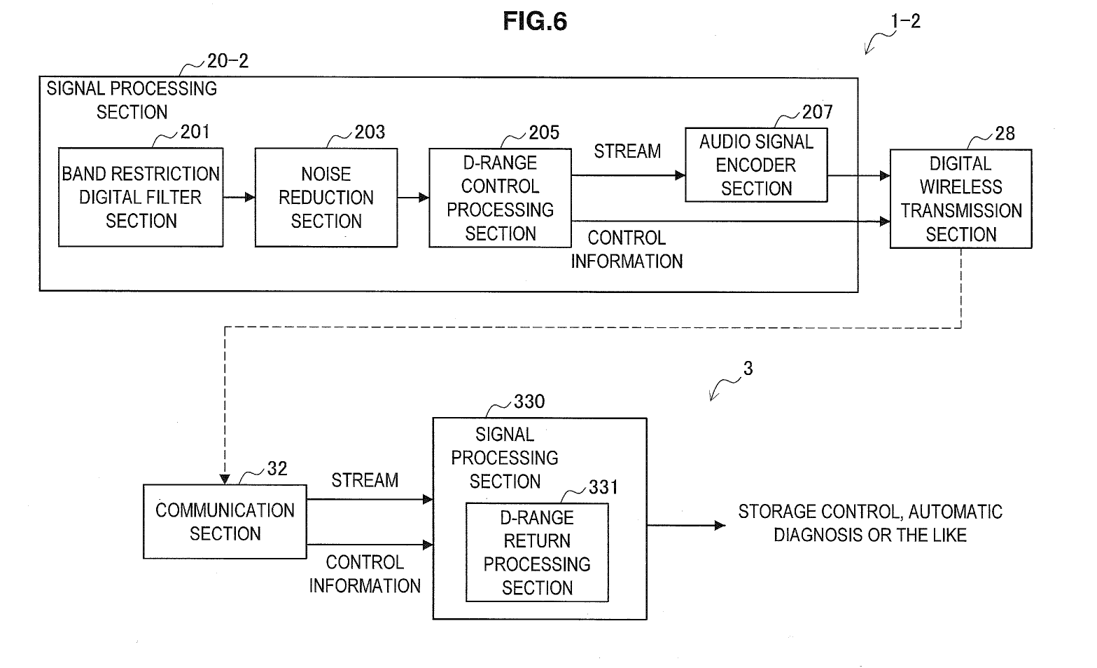

In the example described with reference to FIG. 3, while the signal processing section 20-1 according to the present embodiment has a band restriction digital filter section 201 and an encoder section 207, the configuration of the signal processing section according to the embodiment of the present disclosure is not limited to this. Hereinafter, another configuration example of the signal processing section will be described with reference to FIG. 6.

FIG. 6 is a block diagram for describing another configuration of a signal processing section. As shown in FIG. 6, a signal processing section 20-2 included in the capsule 1-2 may additionally have a noise reduction section 203 and a D-range (Dynamic range) control processing section 205, in order to improve the S/N ratio. Note that in the example shown in FIG. 6, the microphone 10, amplifier 11, and ADC 14 of the capsule 1-2 are omitted.

Further, since the band restriction digital filter section 201 and the encoder section 207 have been described above with reference to FIG. 3, a description of them will be omitted here.

*Noise Reduction Section

The noise reduction (NR) section 203 has a function which cuts a prescribed noise component. In the present embodiment, in the case where a continuous and regular sound, such as blood flow sound, is contained within the collected audio signals at the time when, for example, an observation to be focused on is heart sounds, this blood flow sound is treated as noise, and it is possible to cut the blood flow sound by the NR section 203. Specifically, the NR section 203 may estimate a noise (here, the blood flow sound), based on the results of a sound collection recording and frequency analysis of a certain period, and may use a technique such as SS (Spectrum Subtraction) which subtracts this noise from the audio signal within the observation period on a frequency axis.

*D-Range Control Processing Section

The D-range control processing section 205 has a function which controls the width of the volume of the audio signals output from the NR section 203. In this way, it becomes possible to reduce the load of the processing resources for the encoder section 207 and to reduce the wireless transmission capacity, and the circuit scale and the power consumption of the capsule 1-2 will decrease. Further, since it becomes possible to further reduce the size of the capsule 1-2 by decreasing the circuit scale and the power consumption, an effect will occur in which the burden on a user (test subject) who swallows the capsule 1-2 is reduced.

Further, the D-range control processing section 205 may be implemented by an AGC (Auto Gain Control), a limiter, a compressor or the like.

Note that in the case where the absolute size of the signal itself or a relative change of size in a continuous time becomes important as observation content, there will be cases where the above described D-range control becomes a disadvantage. In preparation for such a case, as shown in FIG. 6, the D-range control processing section 205 according to the present embodiment intermittently outputs control information, which displays the D-range control processing content, separate from stream audio signals output to the audio signal encoder section 207. Such control information is transmitted to the outside by the digital wireless transmission section 28.

Also, as shown in FIG. 3, the stream audio signals and control information transmitted from the digital wireless transmission section 28 are received by the communication section 32 of the control apparatus 3, and are sent to the signal processing section 330. Here, the signal processing section 330 has a D-range return processing section 331, and the D-range return processing section 331 can return the D-range control processed audio signals, based on the control information. In this way, in the present embodiment, sufficient information can be acquired for observation, automatic diagnosis or the like.

Further, even in the case where a return process is not performed, recognizing whether or not there is an effect target of timing by the D-range control processing section 205 (whether or not it is saturated) on the side of the control apparatus 3, for example, is also important from the viewpoint of error sensitivity.

(Change of the Signal Processing Parameters)

Each process of the above described signal processing sections 20-1 and 20-2 (each process of the band restriction digital filter section 201, the NT section 203, the D-range control processing section 205, and the encoder section 207) are not limited to fixed processes, and they may be fluid processes. More specifically, parameters related to each process of the signal processing sections 20-1 and 20-2 may be changed in accordance with the sounds of a part within the body or the sounds of an observation target.

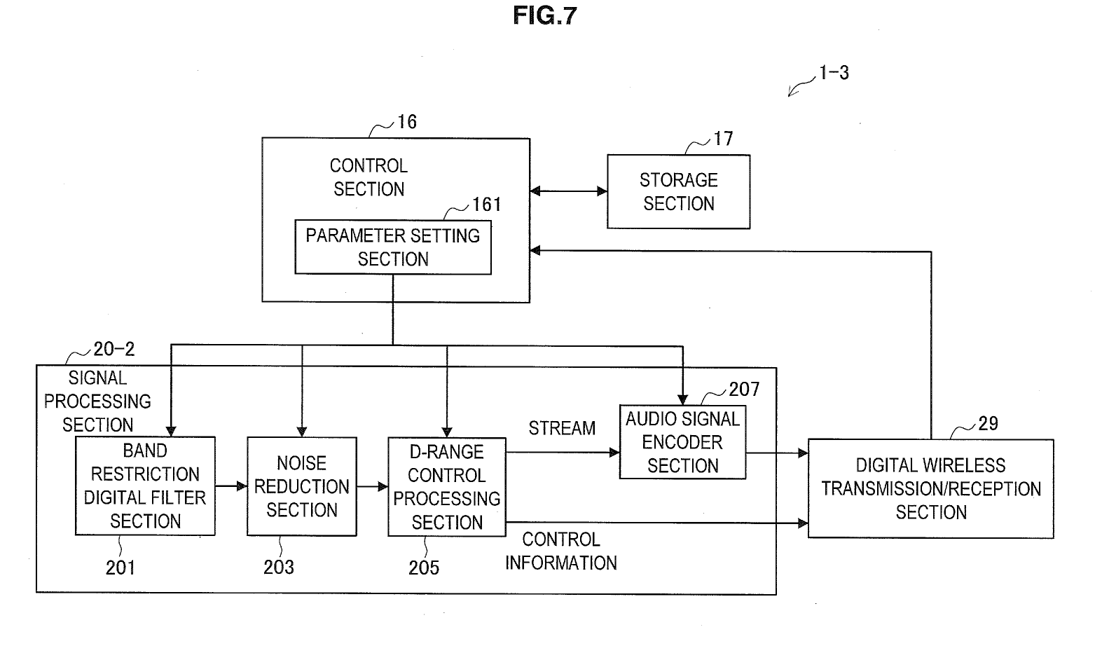

The change of parameters according to the present embodiment may be performed based on instruction signals from the outside, or may be performed inside the capsule. Hereinafter, a capsule 1-3 which changes each parameter based on instruction signals from the outside, and capsules 1-4 and 1-5 which judge and change the parameters internally, will be described with reference to FIGS. 7 to 10.

*Change of Parameters Based on External Controls

FIG. 7 is a block diagram which shows the main constituent elements of the capsule 1-3 according to the present embodiment. As shown in FIG. 7, the capsule 1-3 has a signal processing section 20-2, a digital wireless transmission/reception section 29, a control section 16, and a storage section 17. Note that in the example shown in FIG. 7, the microphone 10, amplifier 11 and ADC 14 of the capsule 1-3 are omitted.

The digital wireless transmission/reception section 29, in addition to a function which transmits and receives audio signals or the like from the outside, has a function which receives data from the outside. The digital wireless transmission/reception section 29 according to the present embodiment receives parameters from the control apparatus 3 (a dedicated device, smart phone, tablet terminal or the like), and outputs the parameters to the control section 16. Further, the received parameters may be stored in the storage section 17, by the control of the control section 16. Further, the storage section 17 may store the audio signals processed by the signal processing section 20-2, by the control of the control section 16.

The control section 16 has a function which controls each constituent element of the capsule 1-3. More specifically, as shown in FIG. 7, the control section 16 functions as a parameter setting section 161. The parameter setting section 161 respectively sets (changes) the parameters in each process of the band restriction digital filter section 201, the NR section 203, the D-range control processing section 205, and the encoder section 207 of the signal processing section 20-2, in accordance with the received parameters. In this way, it becomes possible to execute appropriate processes for the purpose of observation, and S/N improvements can be expected.

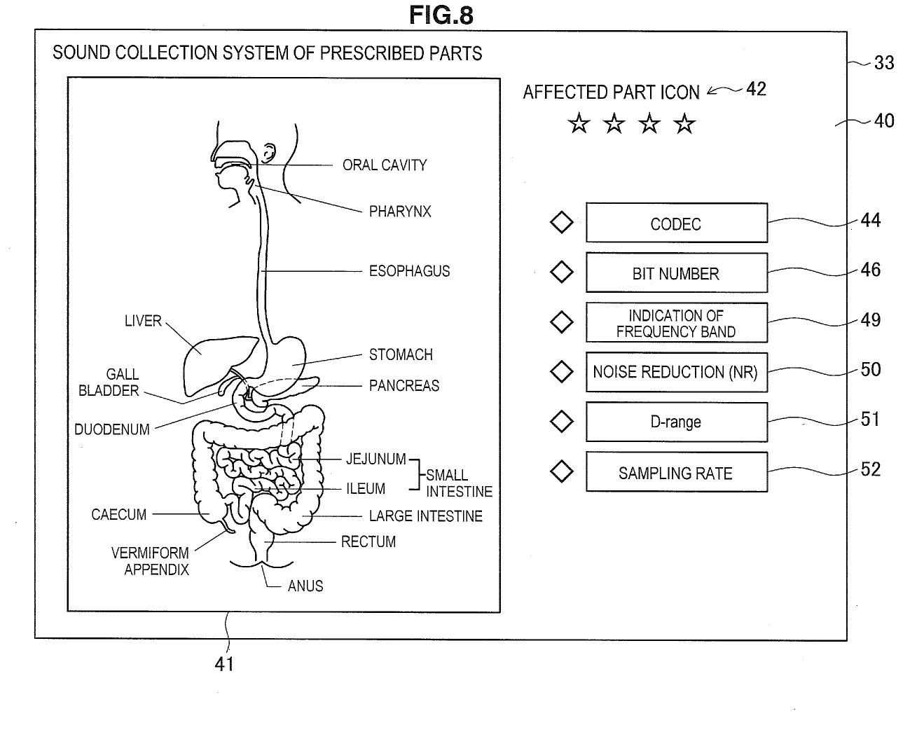

Each parameter set by such external controls for the capsule 1-3 may be determined in accordance with user operations. FIG. 8 is a figure which shows an example of an operation screen displayed on the display section 33 of the control apparatus 3.

As shown in FIG. 8, the operation screen 40 includes a part screen 41, an affected part icon 42, and setting buttons 44 to 52 for setting each observation condition (sound collection method). The part screen 41, as shown in FIG. 8, may be an image in which illustrations and names of each part are associated with each other. The affected part icon 42 is an icon which indicates an observation position (specific part) of sounds, and a user selects, for example, the affected part icon 42 once, moves the affected part icon 42 to an arbitrary position on the part screen 41 by a drag and drop operation, and registers the specific part (refer to step S103 shown in FIG. 5).

In addition, as shown in FIG. 8, a CODEC (encoding and decoding system), a bit number, a restriction of frequency bands, an NR (noise reduction), a D-range, or a sampling rate can be set as the observation condition (sound collection method) of sounds.

Here, in the field of normal stethoscopes which observe sounds within the body from the outside, observation is performed generally by dividing the sounds into 20-200 Hz (bell region), 100-500 Hz (diaphragm region) and the like. It is possible to detect abnormalities mainly of heart murmurs and blood vessel sounds in the bell region, and to detect abnormalities mainly of the respiratory organs in the diaphragm region.

Accordingly, for example, in the sound collection system according to the present embodiment, combinations, such as described hereinafter, are assumed to be set in accordance with the purpose of observation. Note that the combinations shown hereinafter are examples, and the present embodiment is not limited to these, and there may be suitable combinations corresponding to other purposes of observation (an observation position or an observation target).

(a) In the case where the entire body is observed by a wide band . . . Band restriction; 20-8 kHz, NR; Weak, D-range control; Weak, CODEC; ADPCM

(b) In the case where heart sound observation near the heart is intended . . . Band restriction; 20-200 Hz, NR; Strong, D-range control; Weak, CODEC; PCM

(c) In the case of where blood flow sound observation within the blood vessels is intended . . . Band restriction; 100-500 Hz, NR; Weak, D-range control; Strong, CODEC; MP3

In the case where an overall waveform observation may be necessary, such as in the above (a), the band for entire body observation is widened, and those not using much NR or D-range control can easily acquire the intended audio signals. Further, it becomes possible for compression of continuous signals without comparative deterioration, by using ADPCM (Adaptive Differential Pulse Code Modulation) in the encoding.

Further, in the case where heart sounds are an observation target, such as in the above (b), the band restriction for the main by a low region is set to a low region, or the NR for excluding blood flow sounds or the like is strengthened, and the D-range control is weakened so that the variations of size every time there is a heart sound can be observed. Further, since pulsive waveforms such as heart sounds are also important phase information, signal compression is not performed, and here the pulsive waveforms may be acquired from the capsule 1 as PCM (Pulse Code Modulation).

Further, in the case where blood flow sounds are an observation target, such as in the above (c), the band is narrowed to the above diaphragm region, and the NR is set to be weak so that the continuous waveform of the blood flow sounds does not decay. Further, since phase information or the absolute/relative size of a particular waveform is not the point of observation in blood flow sound observation, the D-range control may be set to be strong, or the encoding system may be an MP3 which is irreversibly compressed.

Note that the sampling rate (sampling frequency) may be set, for example, to two or more times that of a necessary frequency band, or bit numbers may individually set the necessary resolutions.

For example, versatility is increased by setting the sampling rate to 44.1 kHz or 48 kHz used by digital audio, and by setting the bit number to 16 bit, 20 bit, 24 bit or the like. In this case, in order to save transmission capacity from the capsule 1, a down sampling process may be performed in the signal processing section 20 (20-1 or 20-2), and this may contribute to resource reductions.

Heretofore, a change of parameters based on external controls has been described. Next, a change of parameters based on internal controls will be described.

*Change of Parameters Based on Internal Controls

The capsule type medical apparatus according to the present embodiment may estimate the present position of the apparatus itself (which part inside the body or within which internal organ it is positioned) and may set (change) the parameters in each process of the signal processing section in accordance with the estimated position.

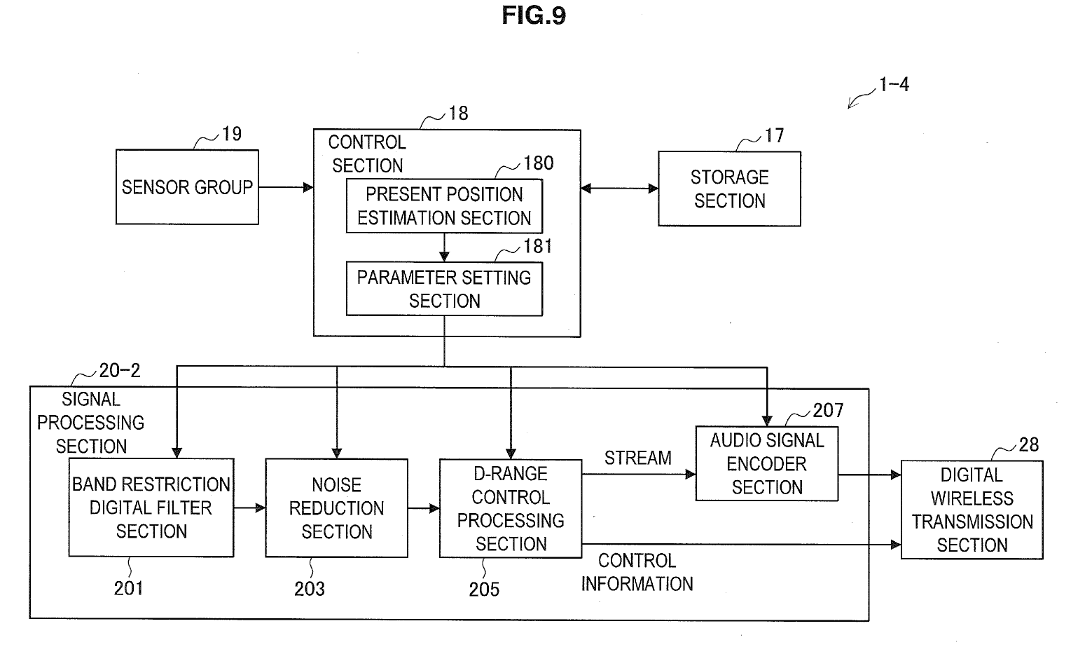

Specifically, for example, as shown in FIG. 9, in the case where the capsule 1-4 according to the present embodiment has a sensor group 19, the present position is estimated based on each sensor value detected from the sensor group 19, and the parameters are set (changed) in each process of the signal processing section 20-2 based on the estimated present position. Note that in the example shown in FIG. 9, the microphone 10, amplifier 11, and ADC 14 of the capsule 1-4 are omitted.

As shown in FIG. 9, the capsule 1-4 has a signal processing section 20-2, a digital wireless transmission section 28, a storage section 17, a control section 18, and a sensor group 19. The storage section 17 may store audio signals processed by the signal processing section 20-2, by the control of the control section 18.

The sensor group 19 may be, for example, pressure sensors, tactile sensors, imaging sensors, acceleration sensors, pH sensors or the like.

The control section 18 functions as a present position estimation section 180 and a parameter setting section 181. The present position estimation section 180 can estimate which part, or within which internal organ, it is presently at, based on each sensor value (pH value or the like) detected by the sensor group 19.

The parameter setting section 181 sets parameters or an encoding system in each process of the band restriction digital filter section 201, the NR section 203, the D-range control processing section 205, and the encoder section 207 of the signal processing section 20-2, based on the estimated present position. Note that the set parameters or the like may be calculated by the parameter setting section 181, or may be set to parameters or the like, which are set by associating each position inside the body cavity in advance, by using a database stored in the storage section 17.

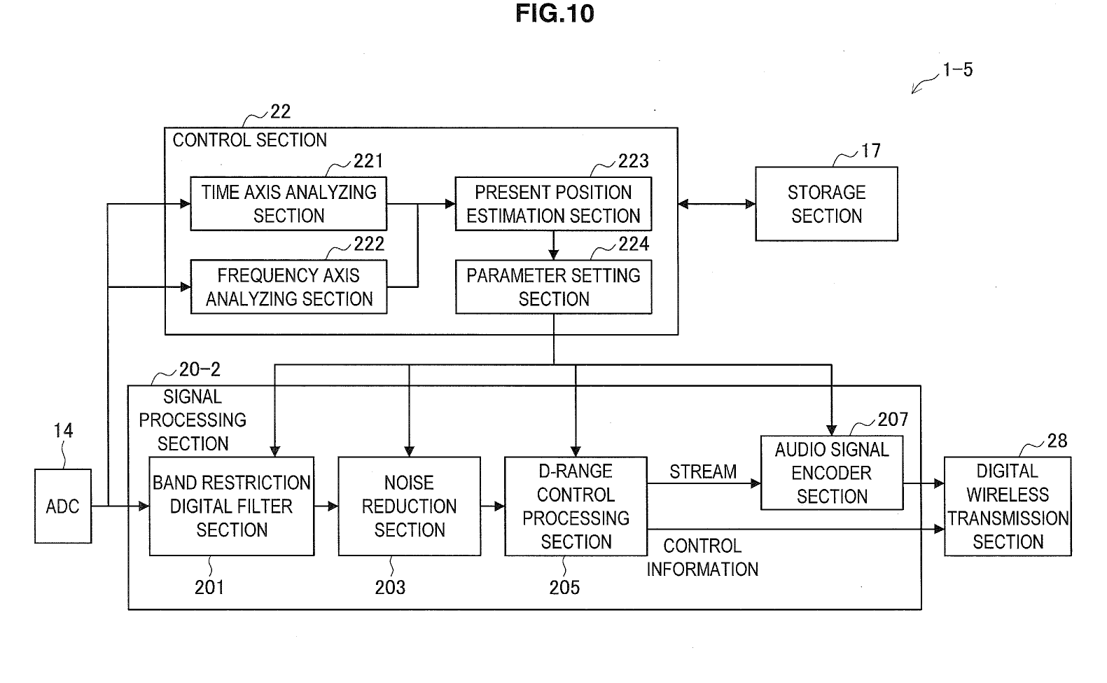

Or, for example, as shown in FIG. 10, the capsule 1-5 according to the present embodiment may analyze the collected body sounds and estimate the present position. Note that in the example shown in FIG. 10, the microphone 10 and amplifier 11 of the capsule 1-5 are omitted.

As shown in FIG. 10, the capsule 1-5 has an ADC 14, a signal processing section 20-2, a digital wireless transmission section 28, a storage section 17, and a control section 22. The storage section 17 may store audio signals processed by the signal processing section 20-2 or estimated present position information, by the control of the control section 22.

The control section 22, as shown in FIG. 10, functions as a time axis analyzing section 221, a frequency axis analyzing section 222, a present position estimation section 223, and a parameter setting section 224. The control section 22 performs control of the audio signals of the collected body sounds output from the ADC 14 so as to analyze the audio signals with a time axis base and a frequency axis base, by using one or both of the time axis analyzing section 221 and the frequency axis analyzing section 222. The time axis analyzing section 221 and the frequency axis analyzing section 222 each output an analysis result (a time axis waveform and a frequency axis waveform) to the present position estimation section 223.

Next, the present position estimation section 223 estimates the present position of the capsule 1, based on each analysis result. More specifically, for example, the present position estimation section 223 may estimate the present position of the capsule 1, by comparing each parameter of the time axis waveform and the frequency waveform, which are associated with each position (part or internal organ) inside the body cavity stored in advance in the storage section 17, with each analysis result.

Also, the parameter setting section 224 sets parameters or an encoding system in each process of the band restriction digital filter section 201, the NR section 203, the D-range control processing section 205, and the encoder section 207 of the signal processing section 20-2, based on the estimated present position.

Note that in the example shown in FIG. 10, each analysis is performed in the control section 22, separate from the processes which are performed by the signal processing section 20-2, and the present position is estimated based on an analysis result. However, the estimation method of the present position according to the present embodiment is not limited to the example shown in FIG. 10, and it is possible, for example, for the capsule 1-5 to estimate the present position by sequentially changing each parameter in the signal processing section 20-2.

More specifically, since an expected signal will be different in accordance with the observation position or observation target, the present position (in the vicinity of a part or internal organ) may be estimated by comparing the acquired audio signals with expected signals stored in storage section 17 in advance by pattern confirmation, or by comparing the error distances of these.

Here, the expected signal may be an expected signal which assumes a case where each parameter corresponding to an observation objective (target), for example, included as the above (b) or (c), is set. Further, the collected audio signals which are compared with the expected signals may be audio signals to which each process is performed by the band restriction digital filter section 201, the NR section 203, and the D-range control processing section 205 of the signal processing section 20-2. In this way, since position estimation can be performed based on audio signals in which noise is controlled, precision can be further improved.

Note that in these operations, information of the type or property (shape of the time wavelength, amplitude or phase by the frequency axis) of the “expected signals” is stored as a DB in the storage section 17 in advance. Also, the capsule 1-5 successively changes each parameter, in a state where the present location is unknown, and compares the audio signals processed under each parameter with the expected signals.

Further, the capsule 1-5 may sequentially set, for example, each parameter which is successively changed, such as shown hereinafter.

Setting 1 . . . Band restriction; 20-8 kHz, NR; Weak, D-range control; Weak

Setting 2 . . . Band restriction; 20-200 Hz, NR; Strong, D-range control; Weak

Setting 3 . . . Band restriction; 100-500 Hz, NR; Weak, D-range control; Strong

Setting 4 . . . Band restriction; 20-200 Hz, NR; Medium, D-range control; Strong

Setting N . . . Band restriction; 100-500 Hz, NR; Strong, D-range control; Medium

Heretofore, a change of parameters based on external/internal controls has been described in detail. To continue, a configuration in the case where the capsule 1 according to the present embodiment has plural types of microphones 10 will be described.

(Plural Types of Microphones)

It is assumed that the capsule type medical apparatus according to the present embodiment collects sounds in various environments, such as the case where the capsule type medical apparatus is present within a liquid such as within blood, or in the case where the capsule type medical apparatus is present within air such as the esophagus. Here, there are microphones which are ideal for air propagation sound collection (for example, electret microphones) and there are microphones which are optimal for underwater propagation sound collection (so called hydrophones) which perform vibration observation by a piezoelectric element, and the specifications of each will be different.

Therefore, a capsule type medical apparatus according to the present embodiment, which is capable of collecting body sounds in different environments, has plural types of microphones, and may determine whether any of the microphones are optimal in accordance with the situation.

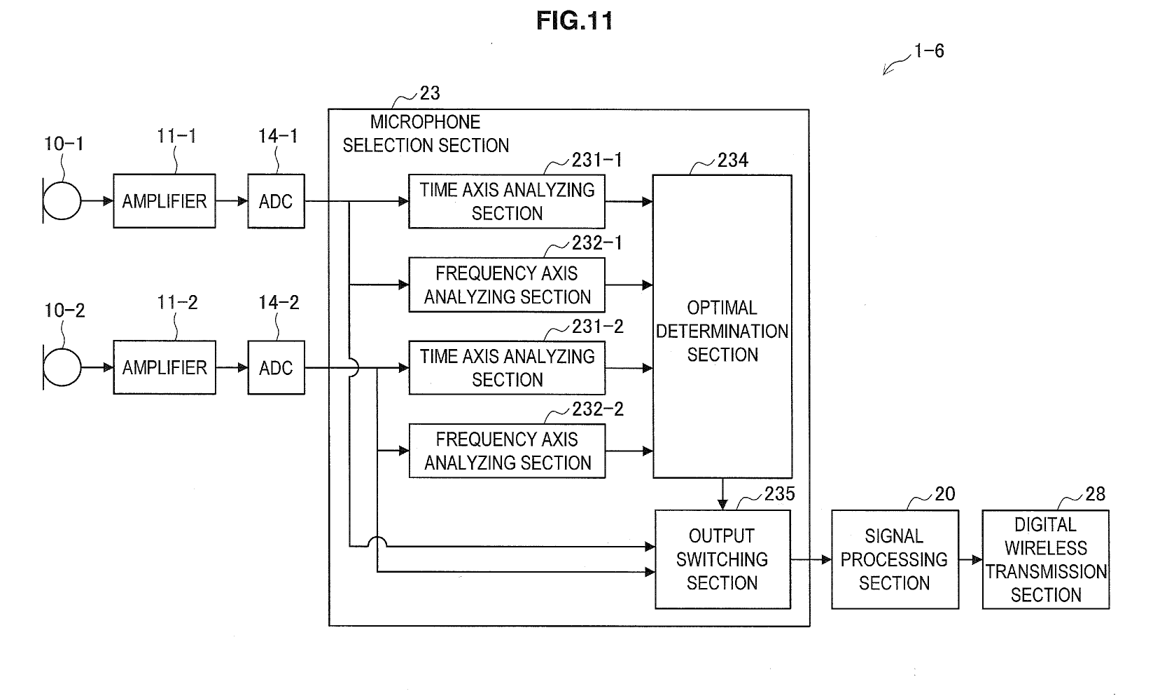

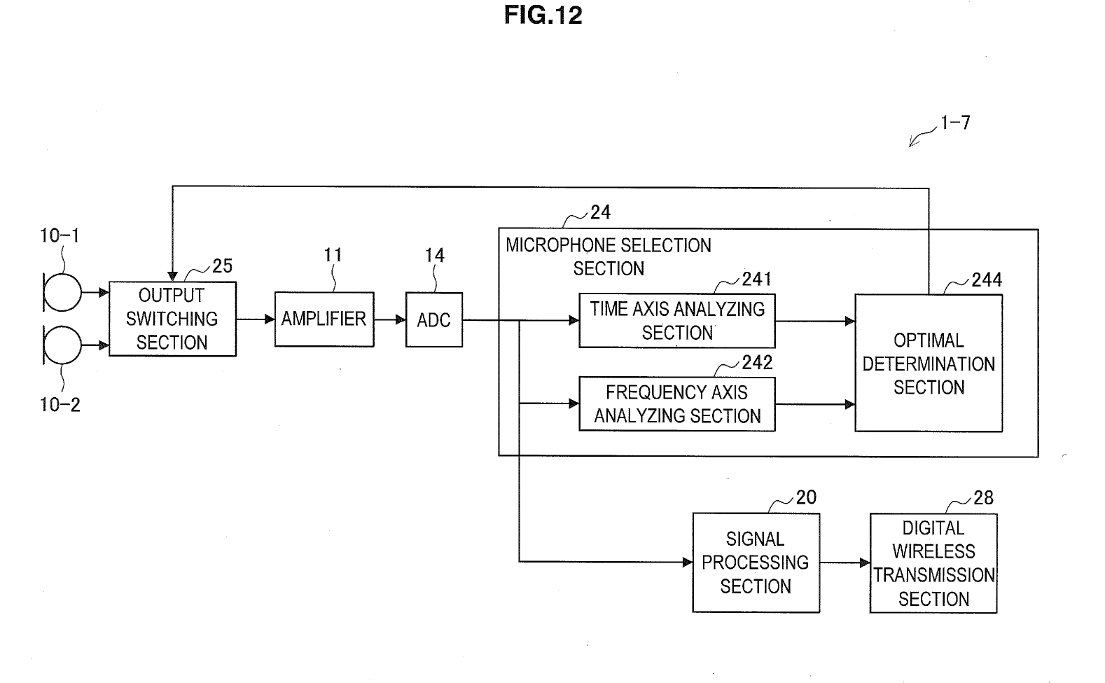

Hereinafter, capsule type medical apparatuses 1-6 (hereinafter, the capsule 1-6) and 1-7 (hereinafter, the capsule 1-7) according to the present embodiment, which have plural types of microphones and which have configurations which determine an optimal microphone, will be described with reference to FIGS. 11 and 12.

*First Selection Method

FIG. 11 is a block diagram which shows the main constituent elements of the capsule 1-6 according to the present embodiment. As shown in FIG. 11, the capsule 1-6 has microphones 10-1 and 10-2, amplifiers 11-1 and 11-2, ADCs 14-1 and 14-2, a microphone selection section 23, a signal processing section 20, and a digital wireless transmission section 28.

The microphones 10-1 and 10-2 are plural types of microphones in which the specifications of each are different. Specifically, for example, the microphone 10-1 may be a microphone optimal for air propagation sound collection, and the microphone 10-2 may be a microphone optimal for underwater propagation sound collection. Note that while two types of microphones are shown here as an example, the present embodiment is not limited to this, and may have, for example, three or more types of microphones.

The microphone selection section 23 has a function which successively analyzes and determines whether one of the microphones 10-1 and 10-2 is optimal. Specifically, as shown in FIG. 11, the microphone selection section 23 has time axis analyzing sections 231-1 and 231-2, frequency axis analyzing sections 232-1 and 232-2, an optimal determination section 234, and an output switching section 235.

The time axis analyzing section 231-1 and the frequency axis analyzing section 232-1 each analyze the audio signals of body sounds collected by the microphone 10-1, and each output an analysis result (a time axis waveform and a frequency axis waveform) to the optimal determination section 234. Further, the time axis analyzing section 231-2 and the frequency axis analyzing section 232-2 each analyze the audio signals of body sounds collected by the microphone 10-2, and each output an analysis result (a time axis waveform and a frequency axis waveform) to the optimal determination section 234.

Next, the optimal determination section 234 determines an optimal microphone from among the microphones 10-1 and 10-2, based on each analysis result. More specifically, for example, the optimal determination section 234 may evaluate the microphones 10-1 and 10-2 from the viewpoints of distortion rate, frequency characteristics, sensitivity or the like, based on each analysis result, and may determine an optimal microphone based on a comprehensive evaluation value.

Then, the output switching section 235 switches the audio signals output to the signal processing section 20, based on a determination result by the optimal determination section 234.

In this way, the microphone selection section 23 according to the present embodiment can successively select audio signals from a microphone determined to be an optimal microphone from among the microphones 10-1 and 10-2, and can output the audio signals to the signal processing section 20 of a later stage.

Note that the signal processing section 20 shown in FIG. 11 may be any one of the above described signal processing sections 20-1 to 20-2.

*Second Selection Method

In the above described example shown in FIG. 11, while determination of which microphone is optimal is performed while successively analyzing all the audio signals output from each microphone, the present embodiment is not limited to this. For example, the capsule type medical apparatus according to the present embodiment may perform optimal determination by switching the audio signals to be analyzed for every time. Hereinafter, a description will be described in detail with reference to FIG. 12.

FIG. 12 is a block diagram which shows the main constituent elements of the capsule 1-7 according to the present embodiment. As shown in FIG. 12, the capsule 1-7 has microphones 10-1 and 10-2, an output switching section 25, an amplifier 11, an ADC 14, a microphone selection section 24, a signal processing section 20, and a digital wireless transmission section 28. Further, the microphone selection section 24 has a time axis analyzing section 241, a frequency axis analyzing section 242, and an optimal determination section 244.

The output switching section 25 first switches the audio signals output to the amplifier 11 for every time. To continue, since the processes of the amplifier 11 and the ADC 14 have been described above with reference to FIG. 3, a description of them will be omitted here.

Next, the microphone selection section 24 performs analysis for the audio signals output from the ADC 14, by each of the time axis analyzing section 241 and the frequency axis analyzing section 242. Each analysis result is output to the optimal determination section 244. Further, each analysis result may be temporarily stored in a storage section (not shown in the figure).

For example, in the case where the audio signals from the microphone 10-1 are output by the output switching section 25, each analysis result for these audio signals are temporarily stored in the storage section. Next, the audio signals from the microphone 10-2 are output by the output switching section 25, and each analysis result for these audio signals are sent to the optimal determination section 244.

To continue, the optimal determination section 244 determines which microphone is an optimal microphone, based on each analysis result for the audio signals of the microphone 10-2 and each analysis result for the audio signals of the microphone 10-1 stored in the storage section (not shown in the figure).

Also, as shown in FIG. 12, when a determination result by the optimal determination section 244 is sent to the output switching section 25, the output switching section 25 performs output switching based on the determination result, and outputs the audio signals determined to be optimal to the amplifier 11.

In this way, the capsule 1-7 can send the audio signals of an optimal microphone to the signal processing section 20. Further, since the configuration of the capsule 1-7 reduces the circuit scale and the processing load more than that of the configuration of the capsule 1-6 shown in FIG. 11, the power consumption will decrease, and it becomes possible to further reduce the size of the capsule.

Heretofore, an optimal determination technique in the case of having plural types of microphones has been described by including two examples. Note that in the case where the present position of the apparatus itself can be estimated, such as described above with reference to FIGS. 9 and 10, the capsule type medical apparatus according to the present embodiment may select an optimal microphone from the plural types of microphones, based on the estimated present position.

### 2-2. The Second Embodiment

In the above described first embodiment, while a case has been described where one capsule is introduced into the body cavity, the sound collection system according to the present embodiment is not limited to this. For example, the sound collection system according to the present embodiment can also be applied to a case where a plurality of capsules are introduced into the body cavity. In this way, more accurate body sounds based on a plurality of audio signals can be acquired, by introducing a plurality of capsules which have one microphone. Hereinafter, a sound collection system according to a second embodiment of the present disclosure, which uses a plurality of capsules, will be described.

[2-2-1. Overall Configuration]

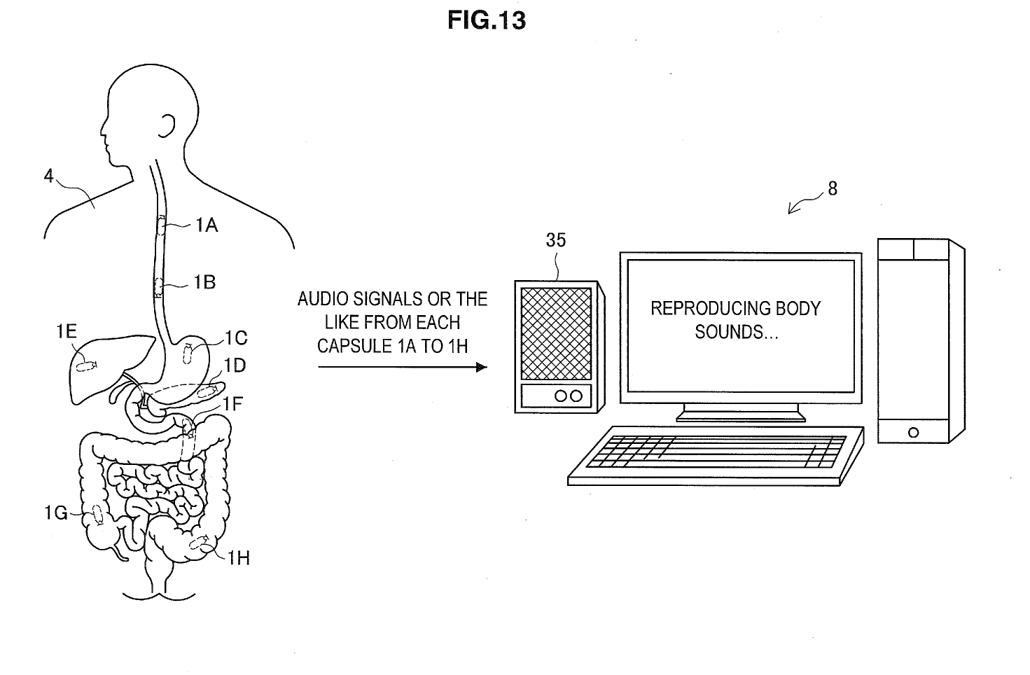

FIG. 13 is a figure for describing the overall configuration of the sound collection system according to the second embodiment. As shown in FIG. 13, the sound collection system according to the present embodiment has a plurality of capsule type medical apparatuses 1A to 1H (hereinafter, called the capsules 1A to 1H) introduced into the body cavity of a test subject 4, and a control apparatus 8.

The configuration of each capsule 1A to 1H may be any one of the capsules 1-1 to 1-7 according to the above described first embodiment.

Further, each capsule 1A to 1H according to the present embodiment has a wireless transmission function, and is capable of transmitting audio signals to the control apparatus 8. For example, while omitted in FIG. 13, in the case where the test subject 4 is fitted with an antenna 5 and an external unit 6 such as shown in FIG. 1, the data transmitted from each capsule 1A to 1H is received by the antenna 5, and is sent to the external unit 6. Then, this data may be transmitted by wires or wirelessly from the external unit 6 to the control apparatus 8.

The control apparatus 8 has a basic configuration similar to that of the control apparatus 3 according to the first embodiment described above with reference to FIGS. 1 and 3, and reproduces body sounds from the speaker 35, for example, based on a plurality of audio signals acquired from each capsule 1A to 1H.

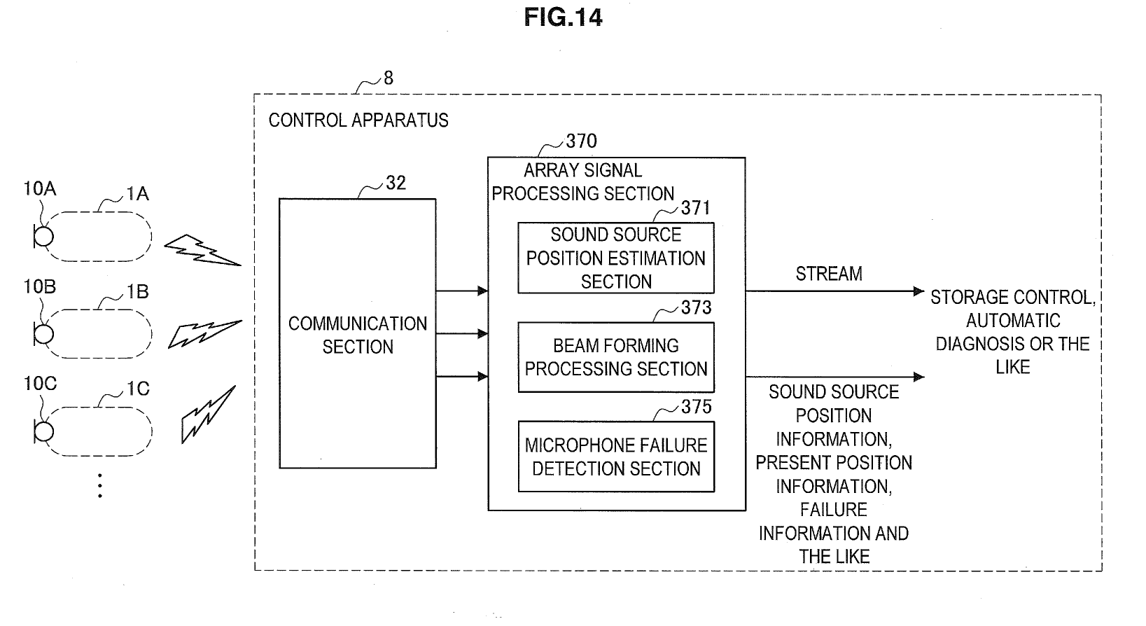

Note that the control apparatus 8 according to the present embodiment may reproduce one or a plurality of the audio signals, from among the plurality of audio signals acquired from each capsule 1A to 1H, and may reproduce a plurality of the audio signals which have been addition processed. Hereinafter, a signal process by the control apparatus 8 will be described with reference to FIG. 14.

[2-2-2. Array Signal Process by the Control Apparatus]

FIG. 14 is a figure for describing an array signal process of the control apparatus 8 according to the second embodiment. Note that in the example shown in FIG. 14, the communication section 32 and the array signal processing section 370 are shown as the main constituent elements of the control apparatus 8, and the other constituent elements (each of the constituent elements excluding the communication section 32 and the signal processing section 330 shown in FIG. 4) are omitted.

(Communication Section)

The communication section 32 receives audio signals or the like from each capsule 1A, 1B, 1C or the like, and outputs the audio signals to the array signal processing section 370.

(Array Signal Processing Section)

The array signal processing section 370 performs a prescribed array signal process for each audio signal acquired from the plurality of capsules 1. Here, the array signal processing section 370 may select audio signals to be signal processed, out of each of the audio signals acquired from the plurality of capsules 1, in accordance with the present position (sound collection location) of each capsule 1.

Further, for example, as shown in FIG. 14, the array signal processing section 370 may function as a sound source position estimation section 371, a beam forming processing section 373, and a microphone failure detection section 375.

*Sound Source Position Estimation Section

The sound source position estimation section 371 estimates the position of a sound source (affected part, abnormal part), based on position information of each capsule and a calculation result of a correlation function of each audio signal. Here, the position information of each capsule may be position information detected based on a signal for position detection originating from the capsule, such as described above in the arrival judgment section 310, or may be present position information estimated in the capsule.

*Beam Forming Processing Section

The beam forming processing section 373 performs a beam forming process in a specific direction from the capsule, based on the position information of each capsule, and can improve the S/N ratio of the audio signals. For example, the beam forming processing section 373 can acquire (generate) more accurate sounds of an affected part (abnormal sounds), by performing a beam forming process in the direction of the sound source (affected part, abnormal part) estimated by the sound source position estimation section 371.

Further, the beam forming processing section 373 may perform a process similar to that of synchronous addition, by using a delay sum array system or the like, and may control random noises. Here, synchronous addition is calculating the data obtained by repeatedly performing the same measurement to match a time axis, and can suppress the influence of noise and improve the S/N ratio by acquiring this average.

*Microphone Failure Detection Section

The microphone failure detection section 375 has a function which detects a microphone failure of each microphone 10A, 10B, 10C or the like of each capsule 1A, 1B, 1C or the like. In this way, a failure diagnosis of a microphone can be set so that audio signals from a failed microphone are not used by the sound source position estimation section 371 or the beam forming processing section 373.

Heretofore, the specific functions of the array signal processing section 370 have been described. As a result of each of the above described processes, the array signal processing section 370 outputs stream audio signals (for example, audio signals to which synchronous addition is performed), sound source position information, present position information, failure information and the like to each of the storage control section 340, the speaker control section 350, and the diagnosis section 360 of a later stage (refer to FIG. 4).

Further, in the present embodiment, since microphones 10 are included in each of the plurality of capsules distributed inside the body cavity, the distances between each microphone are assumed to be separated. In this case, it is possible for the array signal process of the control apparatus 3 to effectively respond to lower frequencies.

Further, in order to accurately perform synchronicity of the audio signals from each capsule, a time code based on a wave clock is added to the audio signals on the side of the capsule 1, and the time of the audio signals from each capsule 1 may be adjusted by buffering on the side of the control apparatus 8.

Note that in the example shown in FIG. 14, while each signal process (band restriction, NR, D-range or the like) for improving the S/N ratio described above with reference to FIGS. 2 to 10 is performed on the side of the capsule 1, the processing resources may be distributed to the side of the control apparatus 8.

### 2-3. The Third Embodiment

In the above described first and second embodiments, the capsule 1 has one (or plural types of one) microphone of the same type. However, the configuration of the capsule type medical apparatus according to the present embodiment is not limited to this, and may have a configuration, for example, which has a plurality of (or plural types of a plurality of) microphones of the same type. Hereinafter, a capsule type medical apparatus 2 (hereinafter, called the capsule 2), which has such a plurality of microphones, will be described in detail as a third embodiment.

[2-3-1. Outline]

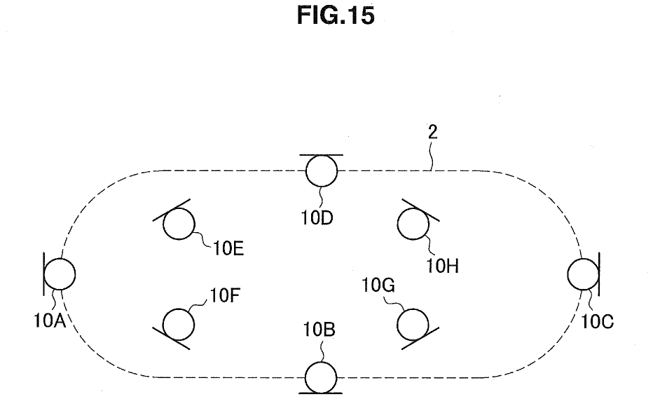

FIG. 15 is a figure for describing an outline of the capsule 2 according to the third embodiment. As shown in FIG. 15, the capsule 2 has a plurality of microphones 10A to 10H. The body sounds collected by each microphone are output as respective audio signals. The capsule 2 according to the present embodiment may reproduce any one or a plurality of the audio signals, from among the plurality of audio signals acquired from each capsule 1A to 1H, or may reproduce a plurality of audio signals which have been addition processed.

[2-3-2. Main Constituent Elements of the Capsule]

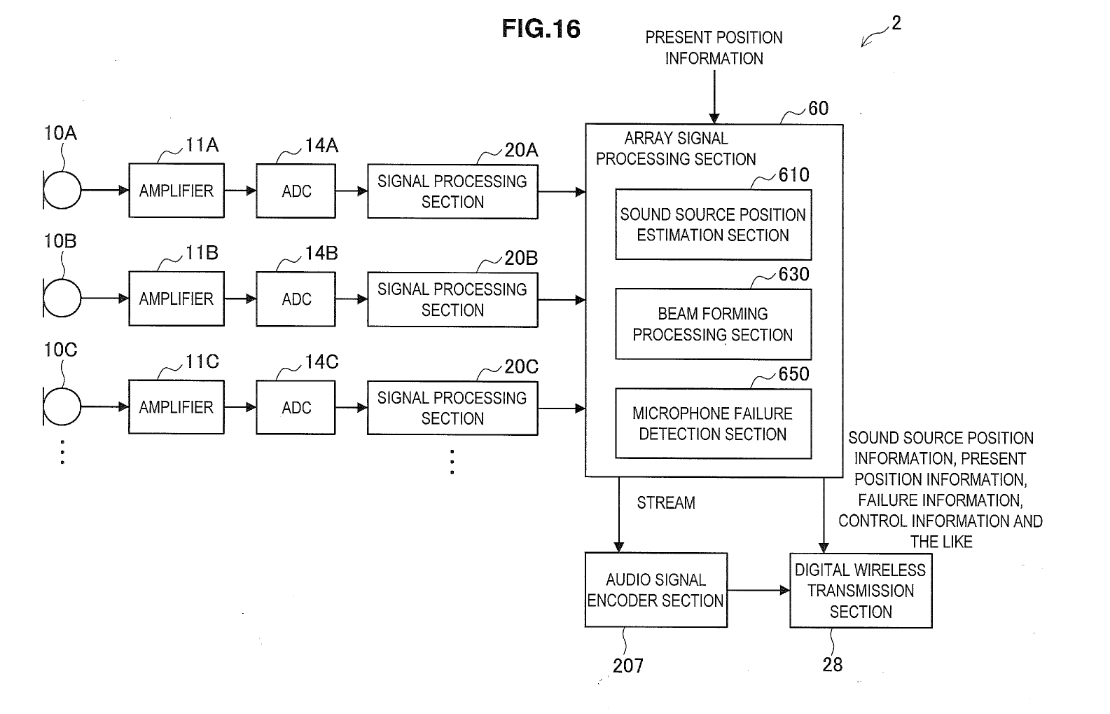

Next, an example of the configuration of the capsule 2 according to the present embodiment will be described with reference to FIG. 16. FIG. 16 is a block diagram which shows the main constituent elements of the capsule 2 according to the third embodiment. As shown in FIG. 16, the capsule 2 has microphones 10A, 10B, 10C, . . . , amplifiers 11A, 11B, 11C, . . . , ADCs 14A, 14B, 14C, . . . , signal processing sections 20A, 20B, 20C, . . . , an array signal processing section 60, an audio signal encoder section 207, and a digital wireless transmission section 28.

As shown in FIG. 16, the audio signals of the body sounds collected by each microphone 10 receive a process by each amplifier 11, ADC 14, and signal processing section 20, and are each output to the array signal processing section 60 which integrally handles each of the signals. Further, present position information of the capsule 2 is input to the array signal processing section 60. Such present position information may be present position information estimated by the above described present position estimation sections 180 and 223.

(Array Signal Processing Section)

As shown in FIG. 16, the array signal processing section 60 functions as a sound source position estimation section 610, a beam forming processing section 630, and a microphone failure detection section 650. Specifically, the sound source position estimation section 610 estimates the position of a sound source (affected part, abnormal part), based on a calculation result of a correlation function of each audio signal.

Further, the beam forming processing section 630 performs a beam forming process in a specific direction from the capsule 2, based on the present position information of the capsule 2, and can improve the S/N ratio of the audio signals. For example, more accurate sounds of an affected part (abnormal sounds) can be acquired (generated), by performing a beam forming process in the direction of the sound source (affected part, abnormal part) estimated by the sound source position estimation section 610.

Further, the microphone failure detection section 650 has a function which detects a microphone failure of each microphone 10A, 10B, 10C and the like. In this way, a failure diagnosis of a microphone can be set so that audio signals from a failed microphone are not used by the sound source position estimation section 610 or the beam forming processing section 630.

Heretofore, the specific functions of the array signal processing section 60 have been described. As a result of each of the above described processes, the array signal processing section 60 outputs stream audio signals (for example, audio signals to which synchronous addition is performed) to the audio signal encoder section 207 of a later stage, and the encoded audio signals are sent to the digital wireless transmission section 28. Further, the array signal processing section 60 may separately output sound source position information, present position information, failure information and the like to the digital wireless transmission section 28 of a later stage.

In this way, in the sound collection system according to the third embodiment, the capsule 2 can acquire more accurate body sounds, by performing an array signal process for the body sounds collected by a plurality of microphones.

In particular, since the distances between each microphone are assumed to be close in the case where a plurality of microphones 10A to 1H are included in one capsule, it is possible for the array signal processing section 60 according to the present embodiment to effectively respond to higher frequencies.

### 2-4. The Fourth Embodiment

Next, a sound collection system will be described in the case where a plurality of the capsules 2, which have a plurality of microphones, according to the above described third embodiment are present inside the body cavity of a test subject 4.

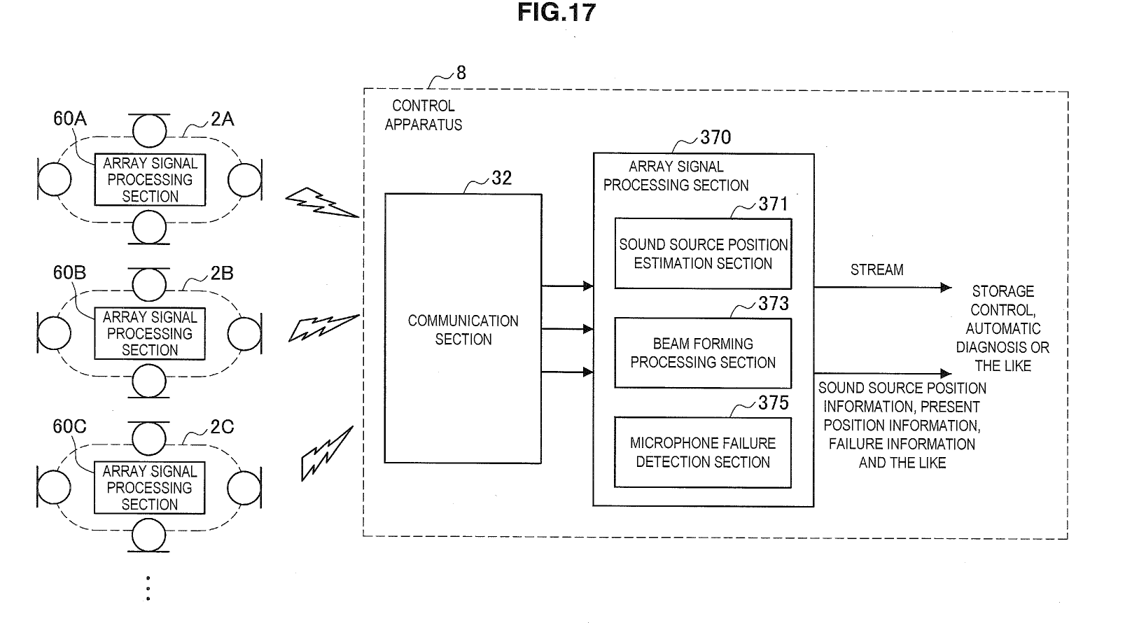

In this case, as shown in FIG. 17, a sound collection system can be implemented by a plurality of capsules, which have a plurality of microphones, and a control apparatus, by combining the control apparatus 8 according to the above described second embodiment with a plurality of capsules 2A to 2C or the like.

The configuration of the plurality of capsules 2A to 2C or the like is similar to the configuration of the capsule 2 according to the above described third embodiment. Therefore, as shown in FIG. 17, each capsule 2A to 2C has array signal processing sections 60A to 60C, respectively, and transmits audio signals to the control apparatus 8 upon performing an array signal process for each audio signal from the plurality of microphones.

On the other hand, as shown in FIG. 17, an array signal process is also performed for each audio signal from the plurality of capsules on the side of the control apparatus 8, by the array signal processing section 370.

In this case, as described above, in the array signal processing section 60 of each capsule 2, since the distances between each microphone are close, it is possible to effectively correspond to higher frequencies. Further, in the array signal processing section 370 of the control apparatus 8, since conversely the distances between each microphone (capsule) are far, it is possible to effectively correspond to lower frequencies.

It is possible to effectively respond to a frequency of a wider band, or to expand an observation target (range) of sounds, by using both of the array signal processing sections 60 and 370 which produce such different characteristics.

Note that an arbitrary user may determine, or the capsule 2 or the control apparatus 8 may automatically determine, whether one or both of the array signal processing sections 60 and 370 are used, in accordance with a frequency band assumed as the body sounds originating from an internal organ or surrounding parts of an observation target.

### 2-5. The Fifth Embodiment

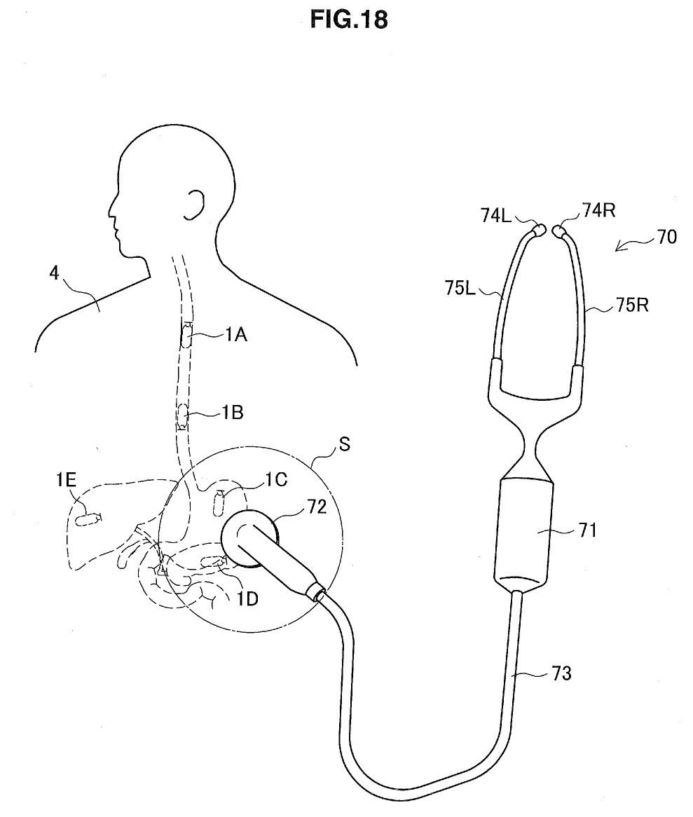

Next, a case will be described with reference to FIG. 18 where body sounds are reproduced by using an electronic auscultation apparatus, in a situation where a plurality of the capsules 1 and 2 are introduced into the body cavity of a test subject 4 described above in the second and fourth embodiments.

FIG. 18 is a figure for describing a sound collection system according to a fifth embodiment of the present disclosure. As shown in FIG. 18, the sound collection system according to the present embodiment has a plurality of capsules 1A to 1E (transmission apparatuses) which have one microphone, and an electronic auscultation apparatus 70 (reception apparatus).

The configuration of the capsules 1A to 1E is similar to that of the capsule 1 according to the above described first and second embodiments. Note that in the example shown in FIG. 18, while the capsule 1, which has one microphone, is used, the present embodiment is not limited to this, and a plurality of the capsules 2, which have a plurality of microphones, according the above described third and fourth embodiments may be introduced into the body cavity of the test subject 4.

The electronic auscultation apparatus 70 has a body section 71, a cable 73, a reception section 72 (sound collection section), ear tube sections 75R and 75L, and ear sections 74R and 74L.

The body section 71 may have an operation input section, a communication section with an external apparatus, a signal processing section, and a storage section or a buffer and the like. Further, the signal processing section (not shown in the figure) may perform a signal process similar to that of the signal processing section 330 or the array signal processing section 370 described above in embodiments 1 to 4. Each audio signal received from the capsules 1 inside the body cavity, or the audio signals to which synchronous addition has been performed by the signal processing section, are stored in the storage section. In this way, it is possible for a plurality of audio signals to be reproduced in the electronic auscultation apparatus 70, or it is possible for the audio signals to be transmitted to the external apparatus afterwards.

The cable 73 is connected to an end section of the body section 71, and the reception section 72 is included at this end section.

The reception section 72 has a function which receives audio signals or the like from the capsules 1 introduced into the body cavity of the test subject 4. Further, the reception section 72 may have the shape of a chest piece, such as shown in FIG. 18.

Further, the reception section 72 according to the present embodiment may receive audio signals from the capsules 1 presently within a range corresponding to the position of the reception section 72, which is positioned outside of the body, from among the one or more capsules 1 introduced into the body cavity. Specifically, for example, as shown in FIG. 18, the reception section 72 receives audio signals from the capsules 1C and 1D inside the body cavity, which are presently within a prescribed range S centered on the reception section 72.

Next, the audio signals received by the reception section 72 are sent to the body section 71 via the cable 73, and a prescribed signal process is performed in the body section 71.

Then, audio signals R output from the body section 71 are reproduced from the ear section 74R included at the end section of the ear tube section 75R, through the ear tube section 75R included so as to extend to the side opposite the side at which the cable 73 of the body section 71 is connected. Further, similarly, audio signals L output from the body section 71 are reproduced from the ear section 74L included at the end section of the ear tube section 75L, through the ear tube section 75L included so as to extend to the side opposite the side at which the cable 73 of the body section 71 is connected.

In this way, a user can listen to the collected body sounds inside body cavity which correspond to the position at which the reception section 72 is applied, from among the one or more capsules introduced into the body cavity, by applying the reception section 72 of the electronic auscultation apparatus 70 to the body surface of the test subject 4. Further, according to the present embodiment, a UX (user experience) is implemented, in which a user can easily specify a specific part (sound collection position) inside the body cavity and can accordingly listen to the collected body sounds, by simply applying the reception section 72 to the body surface of the test subject 4.

### 3. CONCLUSION

As described above, in the sound collection system according to the embodiments of the present disclosure, it becomes possible to more effectively acquire body sounds used for diagnosis. For example, the capsule according to the embodiment of the present disclosure can store body sounds collected inside the body cavity within the capsule.

Further, in the sound collection system according to the first embodiment, a capsule 1, which has one microphone 10, performs a band restriction filter process or the like for body sounds collected from the one microphone 10, and can transmit more accurate body sounds to a control apparatus 3.

Further, in the sound collection system according to the second embodiment, it is possible to more effectively respond to audio signals (body sounds) of high frequencies, by performing an array signal process for each audio signal transmitted from a plurality of capsules 1.

Further, in the sound collection system according to the third embodiment, it is possible to more effectively respond to audio signals (body sounds) of low frequencies, by performing an array signal process for body sounds collected by a capsule 2, which has a plurality of microphones 10, from the plurality of microphones 10.

Further, in the sound collection system according to the fourth embodiment, it is possible to more effectively respond to frequencies of a wider band, in the case where a plurality of capsules 2, which have a plurality of microphones 10, are introduced into the body cavity, by performing an array signal process by both the capsules 2 and a control apparatus 8.

Further, in the sound collection system according to the fifth embodiment, it is possible to listen to body sounds collected at the position inside the body cavity which corresponds to a reception section 72, in the case where one or more capsules 1 and 2 are introduced into the body cavity, by applying the reception section 72 of an electronic auscultation apparatus 70 to the body surface of a test subject 4.

It should be understood by those skilled in the art that various modifications, combinations, sub-combinations and alterations may occur depending on design requirements and other factors insofar as they are within the scope of the appended claims or the equivalents thereof.

For example, in the first embodiment, while the vicinity of a specific part is registered in advance, and body sounds are collected in the case where the capsule 1 reaches the vicinity of the specific part, the present embodiment is not limited to this, and the capsule 1 may continuously collect body sounds.

Further, the capsules 1 and 2 according to the present embodiment may have a stop section (not shown in the figures), and may stop in the vicinity of a specific part or at a location diagnosed as an abnormal part and continuously perform observations of body sounds. Note that there are various implementation methods of the stop section, and may be, for example, an arm type such as disclosed in JP 2005-204806A, or may be a balloon type such as disclosed in JP 2003-325438A. An arm type is a stop method which remains inside the body cavity by holding the mucous membrane on the body cavity inner wall with a plurality of arms. Further, a balloon type is a stop method which remains inside the body cavity by expanding a balloon, which has an airtight function with free expansion/contraction included so as to cover the outer surface of part of the capsule, with pressurized gas stored inside the capsule.

Further, in the each of the above described embodiments, while a capsule type medical apparatus is used as an example of a medical apparatus introduced into the body cavity, the medical apparatus according to the embodiment of the present disclosure is not limited to this, and may be, for example, an endoscope in which at least a part is introduced inside the body cavity of a test subject 4.

Additionally, the present technology may also be configured as below:

(1) A storage control apparatus including:

a detection section which detects a body sound inside a body cavity, and outputs the body sound as an audio signal; and

a storage control section which performs control in a manner that the audio signal output from the detection section is stored.

(2) The storage control apparatus according to (1), further including:

a storage section,

wherein the storage control section performs control in a manner that the audio signal is stored in the storage section.

(3) The storage control apparatus according to (1) or (2), further including:

a transmission section which transmits the audio signal to an external apparatus,

wherein the storage control section temporarily stores the audio signal for transmission by the transmission section.

(4) The storage control apparatus according to any one of (1) to (3),

wherein, in a case of reaching a vicinity of a specific part inside the body cavity, the storage control section performs control in a manner that the audio signal is recorded.

(5) The storage control apparatus according to any one of (1) to (4), further including:

a filter section which performs processing in a manner that a prescribed frequency band of the audio signal is extracted.

(6) The storage control apparatus according to any one of (1) to (5), further including:

a noise reduction section which performs processing in a manner that noise of the audio signal is reduced.

(7) The storage control apparatus according to any one of (1) to (6), further including:

a D-range control processing section which performs processing in a manner that dynamic range control of the audio signal is performed.

(8) The storage control apparatus according to any one of (1) to (7), further including:

an encoder section which performs processing in a manner that the audio signal is encoded.

(9) The storage control apparatus according to any one of (5) to (8), further including:

a setting section which sets prescribed a parameter when performing processing for the audio signal.

(10) The storage control apparatus according to any one of (1) to (7), further including:

a plurality of the detection sections.

(11) The storage control apparatus according to (10), further including:

an array signal processing section which processes the audio signal output from each of the plurality of detection sections.

(12) The storage control apparatus according to any one of (1) to (11), further including:

a stop section for stopping in a vicinity of a specific part inside the body cavity.

(13) The storage control apparatus according to any one of (1) to (12), further including:

an imaging section which images inside the body cavity.

(14) The storage control apparatus according to any one of (1) to (12),

wherein the storage control apparatus is a capsule type medical apparatus introduced into the body cavity.

(15) A storage control system including:

a transmission apparatus including
a detection section which detects a body sound inside a body cavity, and outputs the body sound as an audio signal, and
a transmission section which transmits the audio signal output from the detection section to an external apparatus after being temporarily stored; and

a reception apparatus including
a reception section which receives the audio signal from the transmission apparatus, and
a storage control section which performs control in a manner that the audio signal received by the reception section is stored.
   (16) The storage control system according to (15),

wherein the reception apparatus further includes

a reproduction section which reproduces the temporarily stored audio signal by control of the storage control section.

(17) The storage control system according to (15) or (16),

wherein, from among one or more of the transmission apparatuses introduced into the body cavity, the reception section receives the audio signal from the transmission apparatuses within a range corresponding to a position of the reception section outside of the body.

(18) The storage control system according to any one of (15) to (17),

wherein the reception apparatus further includes

an array signal processing section which processes the audio signal received from each of one or more of the transmission apparatuses.

(19) The storage control system according to any one of (15) to (18),

wherein the transmission apparatus is a capsule type medical apparatus introduced into the body cavity.

(20) A storage medium having a program stored thereon, the program causing a computer to function as:

a detection section which detects a body sound inside a body cavity, and outputs the body sound as audio signals; and

a storage control section which performs control in a manner that the audio signal output from the detection section is stored.

The present disclosure contains subject matter related to that disclosed in Japanese Priority Patent Application JP 2012-152087 filed in the Japan Patent Office on Jul. 7, 2012, the entire content of which is hereby incorporated by reference.

What is claimed is:

1. A storage control apparatus comprising:
a detection section which detects a body sound inside a body cavity, and outputs the body sound as an audio signal; and
a storage control section which performs control in a manner that the audio signal output from the detection section is stored.

2. The storage control apparatus according to claim 1, further comprising:
a storage section,
wherein the storage control section performs control in a manner that the audio signal is stored in the storage section.

3. The storage control apparatus according to claim 1, further comprising:
a transmission section which transmits the audio signal to an external apparatus,
wherein the storage control section temporarily stores the audio signal for transmission by the transmission section.

4. The storage control apparatus according to claim 1,
wherein, in a case of reaching a vicinity of a specific part inside the body cavity, the storage control section performs control in a manner that the audio signal is recorded.

5. The storage control apparatus according to claim 1, further comprising:
a filter section which performs processing in a manner that a prescribed frequency band of the audio signal is extracted.

6. The storage control apparatus according to claim 1, further comprising:
a noise reduction section which performs processing in a manner that noise of the audio signal is reduced.

7. The storage control apparatus according to claim 1, further comprising:
a D-range control processing section which performs processing in a manner that dynamic range control of the audio signal is performed.

8. The storage control apparatus according to claim 1, further comprising:
an encoder section which performs processing in a manner that the audio signal is encoded.

9. The storage control apparatus according to claim 5, further comprising:
a setting section which sets prescribed a parameter when performing processing for the audio signal.

10. The storage control apparatus according to claim 1, further comprising:
a plurality of the detection sections.

11. The storage control apparatus according to claim 10, further comprising:
an array signal processing section which processes the audio signal output from each of the plurality of detection sections.

12. The storage control apparatus according to claim 1, further comprising:
a stop section for stopping in a vicinity of a specific part inside the body cavity.

13. The storage control apparatus according to claim 1, further comprising:
an imaging section which images inside the body cavity.

14. The storage control apparatus according to claim 1,
wherein the storage control apparatus is a capsule type medical apparatus introduced into the body cavity.

15. A storage control system comprising:
a transmission apparatus including
a detection section which detects a body sound inside a body cavity, and outputs the body sound as an audio signal, and
a transmission section which transmits the audio signal output from the detection section to an external apparatus after being temporarily stored; and
a reception apparatus including
a reception section which receives the audio signal from the transmission apparatus, and
a storage control section which performs control in a manner that the audio signal received by the reception section is stored.

16. The storage control system according to claim 15,
wherein the reception apparatus further includes
a reproduction section which reproduces the temporarily stored audio signal by control of the storage control section.

17. The storage control system according to claim 15,
wherein, from among one or more of the transmission apparatuses introduced into the body cavity, the reception section receives the audio signal from the transmission apparatuses within a range corresponding to a position of the reception section outside of the body.

18. The storage control system according to claim 15,
wherein the reception apparatus further includes
an array signal processing section which processes the audio signal received from each of one or more of the transmission apparatuses.

19. The storage control system according to claim 15,
wherein the transmission apparatus is a capsule type medical apparatus introduced into the body cavity.

20. A storage medium having a program stored thereon, the program causing a computer to function as:
a detection section which detects a body sound inside a body cavity, and outputs the body sound as audio signals; and
a storage control section which performs control in a manner that the audio signal output from the detection section is stored.
